(19) United States
(12) Patent Application Publication
(10) Pub. No.: US 2012/0014934 A1
(43) Pub. Date: Jan. 19, 2012

(54) Enhanced Natural Colors

(76) Inventors: Paul Altaffer, Alameda, CA (US); Jeffrey M. Wuagneux, Blauvelt, NY (US); Pi-Yu Hsu, Alpine, NJ (US)

(21) Appl. No.: 13/181,878

(22) Filed: Jul. 13, 2011

Related U.S. Application Data
(60) Provisional application No. 61/363,830, filed on Jul. 13, 2010.
(60) Provisional application No. 61/410,621, filed on Nov. 5, 2010.

(51) Int. Cl. A61K 38/43; A61K 31/20; A61K 38/00; A61K 31/70; A61K 36/53; A61K 31/05; A61K 36/752; A61K 31/194; A61K 31/19; A61K 31/122; A61K 31/56; A61K 31/715; A61K 31/522; A61K 31/44; A61K 31/51; A61K 31/195; A61K 31/405; A61K 36/00; A61K 31/202; A61K 31/685; A61K 36/16; A61P 39/06; A61P 29/00; A61K 33/00

(52) U.S. Cl. 424/94.1; 424/600; 514/558; 514/1.1; 514/23; 424/745; 514/731; 424/736; 514/574; 514/557; 514/690; 514/733; 514/182; 514/54; 514/263.34; 514/300; 514/276; 514/565; 514/567; 514/419; 424/725; 514/560; 514/78; 424/752

(57) ABSTRACT

A natural color is concentrated to intensify color range and to provide useful amounts of one or more of anti-oxidant, nutritional, and anti-inflammatory compounds derived from one or more pigment sources. In a preferred embodiment, the pigment source is a fruit, a vegetable, a legume, a spice, algae, or a combination thereof.

### CROSS-REFERENCE TO RELATED DOCUMENTS

The present invention claims priority to a U.S. provisional patent application Ser. Nos. 61/410,621, filed Nov. 5, 2010, and 61/363,830, filed Jul. 13, 2010, both of which are entitled “Enhanced Natural Colors That Provide Color and Nutritional Properties”. The specification also includes accompanying appendices A, B, C and D.

### BACKGROUND OF THE INVENTION

1. Field of the Invention

The present invention is in the field of processing natural pigments and pertains particularly to methods and apparatus for concentrating and enhancing natural pigments to include useful amounts of anti-oxidants and anti-inflammatory agents.

2. Discussion of the State of the Art

Various home-based and commercial processes exist for generating natural pigments from organic materials including fruits, vegetables, legumes, and spices. Natural pigmentation is used in state-of-art processes related to the generation of compounds that include the natural pigment as a desired color. Artificial coloring has been used extensively in a wide variety of products, however natural pigments are gaining recognition as being a healthier alternative to artificial coloring.

Oxidative Stress and Inflammation are two of the most important markers for disease states and are associated with many illnesses and dysfunctions. Anti-oxidants and nutrients are therefore important regimens in disease treatment and prevention.

Natural colors are comprised mostly of classes of ingredients considered to be antioxidants. Products with natural pigments include food and beverage products, dietary supplements, pharmaceuticals, skin care and cosmetics, and similar compounds that require some form of pigmentation in the mix of compounds and ingredients. Current state-of-art processes for generating these natural pigments focus on preserving the color in the compound as opposed to preserving amounts of nutrients and/or anti-oxidative components that may also be found in the pigment source.

A limitation with current natural pigment processing techniques is that generating the color is the primary focus of generating the compounds. As a result, consumer products bearing natural pigments have nutritional and anti-oxidation properties that are not useful in the compound because the concentration levels are too low. These natural pigment compounds would need to be consumed at a very high rate in order that nutritional and anti-oxidant properties have any positive effect on the consumer. It would be desired that natural pigments used in consumer products include useful levels of the nutrients and anti-oxidants that are generic to the pigment sources.

Therefore, what is clearly needed is a set of natural colors that include higher or useful levels of source-associated and anti-oxidants and nutrients while preserving, and in many cases, improving the intensity of the associated color, as well as preserve the functionality as a color, like flavor and aroma neutrality.

### SUMMARY OF THE INVENTION

The problem stated above is that nutritional, anti-inflammatory, and anti-oxidative value is desirable for natural color compounds, but many of the conventional means for extracting natural colors, such as a screw press, only focus on obtaining the color. The inventors discovered that by using various methods for extraction, purification, and concentration, these natural color compounds could be enhanced significantly to include useful amounts of vitamins, nutrients, antioxidants, and anti-inflammatory elements and compounds.

The present inventor realized in a moment of discovery that, at the point of extraction, natural color compounds could be caused to exhibit useful amounts of anti-oxidants, anti-inflammatory compounds, and nutrients either derived from the source materials or caused to be retained within the compound from an external source separate from the original compound. The nutritive compounds can be concentrated to a point where they exert a function beyond color, into the area of human nutrition, while preserving the functional properties of the natural color.

The invention discloses processes and actual product examples that accomplish both goals of preserving functional color and nutritional ingredients. The processes and products illustrated have all been demonstrated to have both highly concentrated functional color properties with acceptable use characteristics (like little flavor or aroma impact), as well as known nutritive qualities, primarily as anti-oxidants and anti-inflammatory properties.

Accordingly, in one embodiment of the present invention, a natural color is concentrated to intensify color range and to provide useful amounts of one or more of anti-oxidant, nutritional, and anti-inflammatory compounds derived from one or more pigment sources. In a preferred embodiment, the pigment source is a fruit, a vegetable, a legume, a spice, algae, or a combination thereof.

In one embodiment, the pigment or pigments in the color are extracted from or concentrated from one or a combination of grape, beet, red cabbage, red radish, hibiscus, carmine, red sandalwood, purple carrot, black carrot, purple sweet potato, purple corn, black currant, bilberry, elderberry, maqui berry, natural carotenoids, carrot, turmeric, curcumin, paprika, annatto, lutein, marigold, spinach, chlorophyll, and spirulina. In one embodiment, the pigment or pigments in the color are extracted by one of or a combination of screw press, hydraulic press, juicing, natural solvent extraction, synthetic solvent extraction, and distillation.

In a variation of this embodiment, the natural color is further processed by one or a combination of vacuum concentration, steam concentration, supercritical carbon dioxide extraction, distillation, ultra-filtration, membrane filtration, column purification, and ion exchange. In one embodiment, the color compound is dried using one or a combination of spray drying, vacuum drying, drum drying, refractance window drying, radiant zone drying and freeze drying.

In one embodiment, the color compound is endogenous. In another embodiment, the color compound is exogenous. In a variation of the endogenous embodiment, the color compound includes one or more of vitamins, minerals, fats, proteins, and sugars. In a variation of the exogenous embodiment, the color compound includes one or a combination of rosemary, butylated hydroxytoulene (BHT), citrus oils, citric acid, and potassium sorbate. In another variation of the exogenous embodiment, the color compound includes one or more of the compounds co-enzyme Q10 (CoQ10), resveratrol, statins, phytosterols, and dietary fiber.

In yet another variation of the exogenous embodiment, the color compound includes one or more of polysaccharides, methylxanthine, caffeine, theobromine and theophylline. In another variation of this embodiment, the color compound includes one or more of 1-thiamine, 1-arginine, 1-phenylalanine, 1-tryptophan, rhodiola, and rosea. In a further variation to this embodiment, the natural color compound includes one or more of omega 3 fatty acids, docosahexanoic acid (DHA), eicosapentaenoic acid (EPA), phosphatidyl choline, phosphatidyl serine, and gingko biloba.

### BRIEF DESCRIPTION OF THE DRAWING FIGURES

FIG. 1 is an organizational chart 100 listing phenolic compounds and their sources.

FIG. 2 is an organizational chart 200 listing vitamins and their sources.

FIG. 3 is an organizational chart 300 listing carotenoid terpenoids and their sources.

FIG. 4 is a collection of organizational charts 400 listing vitamin co-factors and minerals, organosulpher compounds, and hormones and organic antioxidants and their sources.

### DETAILED DESCRIPTION

The inventors have discovered that natural colors may be enhanced through a variety of extraction and or concentration methods to include useful amounts of one or more of anti-oxidant, nutritional, and anti-inflammatory compounds derived from one or more pigment sources. The present invention will be described in enabling detail using the following examples, which may describe more than one relevant embodiment falling within the scope of the present invention.

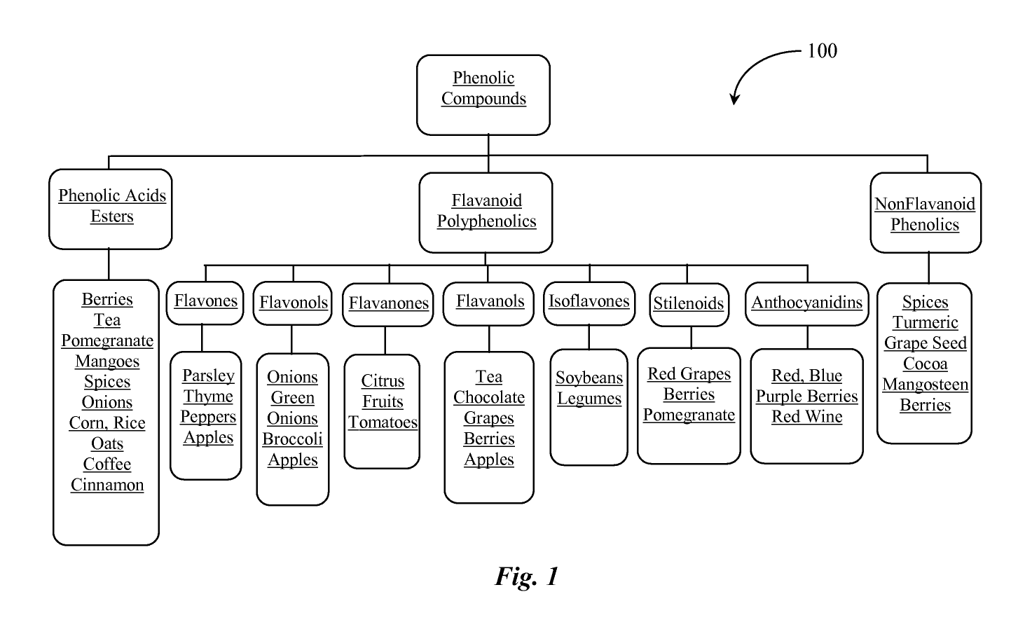

FIG. 1 is an organizational chart 100 listing phenolic compounds and their sources.

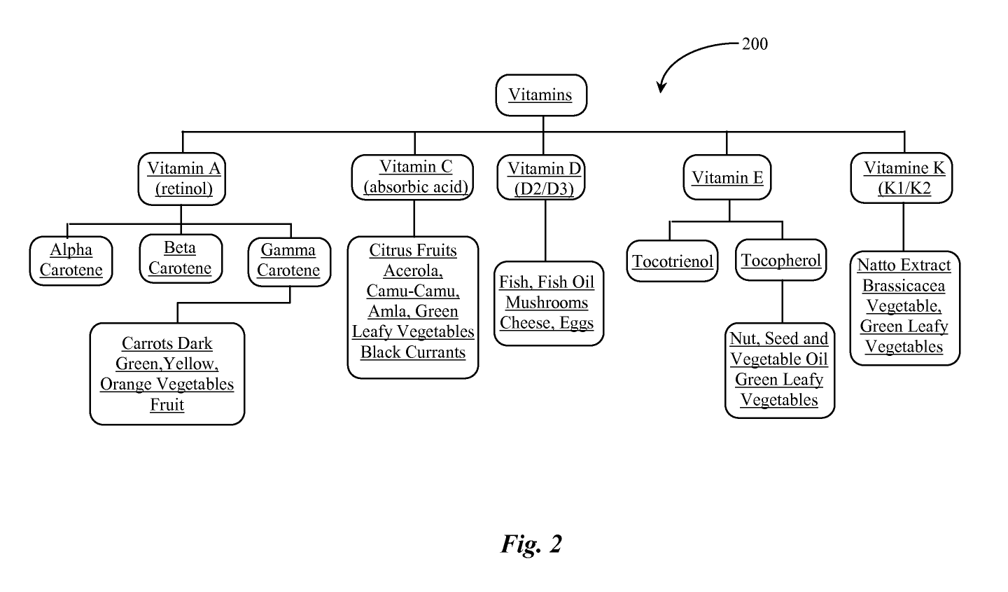

FIG. 2 is an organizational chart 200 listing vitamins and their sources.

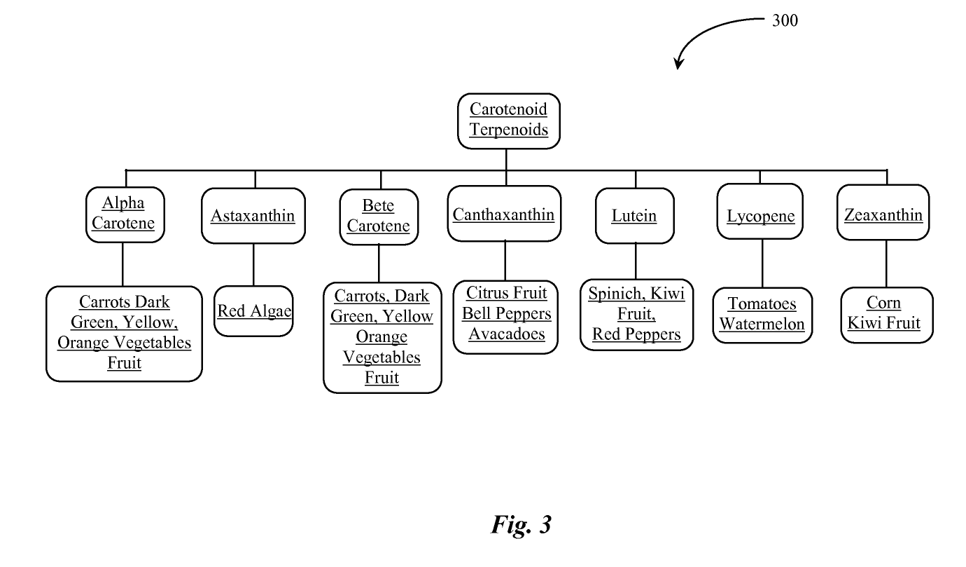

FIG. 3 is an organizational chart 300 listing carotenoid terpenoids and their sources.

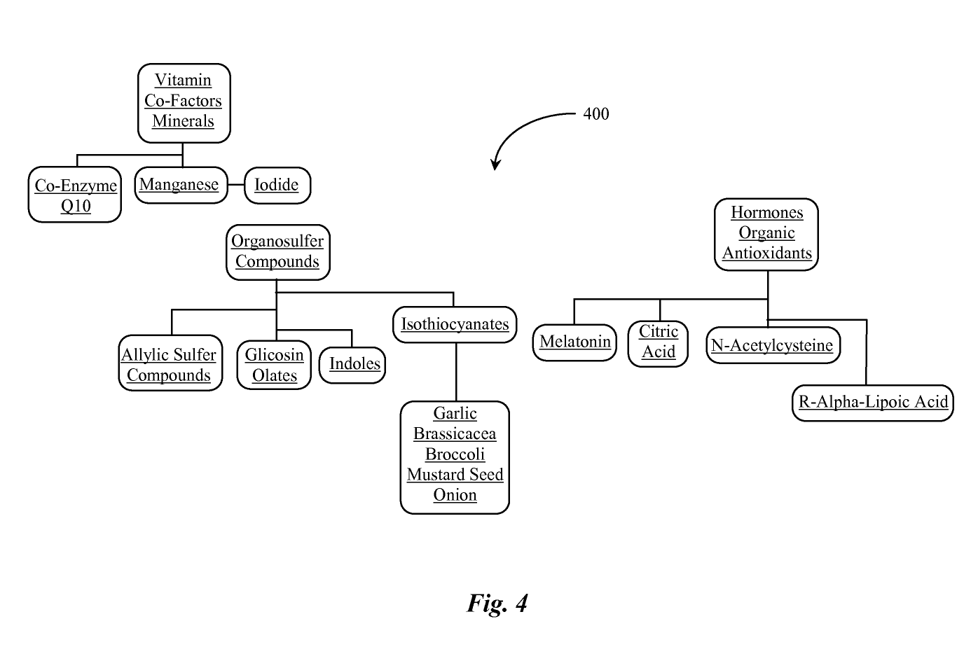

FIG. 4 is a collection of organizational charts 400 listing vitamin co-factors and minerals, organosulpher compounds, and hormones and organic antioxidants and their sources.

### Manufacturing Methods

Referring now to FIGS. 1-4, the enhanced composition (natural color) can be achieved through a variety of manufacturing and concentrating techniques. Typically speaking, natural colors are extracted from a variety of natural ingredients including (but not limited to) berries, grapes, carrots (orange and black/purple), beets, purple sweet potato, red cabbage, red radish, purple corn, hibiscus, different marine algae (including spirulina), paprika, marigold, lutein, annatto, tomato, turmeric and spinach. The natural colors can be extracted from whole products, juices or even waste streams. Natural colors are often extracted using techniques like pressing (screw press, hydraulic press and so on), juicing, solvent extraction (natural and synthetic solvents), distillation as well as supercritical carbon dioxide. The enhanced composition goes beyond these processing techniques and includes additional processes, including: Concentration (vacuum, steam), Supercritical CO2 (carbon dioxide) extraction and purification, Distillation, Ultra-Filtration, Membrane Filtration, Column or Purification, Ion Exchange Resins. Similarly, the enhanced composition can be dried and appropriate technologies for drying include: spray drying, vacuum drying, drum drying, refractance window drying, radiant zone drying and freeze drying.

### Families of Antioxidant Compounds

Referring now to FIGS. 1-4, while almost all natural colors contain antioxidant and/or anti-inflammatory pigments, not all antioxidants are pigments. The discovery focuses on a select range of antioxidant compounds that have color as well as nutritional value. These families of compounds include, but are not limited to:

| Class of | | | | Food/Supplement |
|---|---|---|---|---|
| Compounds | Sub-Class A | Sub-Class B | Compound | Source |
| Phenolic | | | | |
| Compounds | | | | |
| | Flavanoid | | | |
| | Polyphenolics | | | |
| | | Flavones | | Parsley, Thyme, |
| | | | | Peppers, Apples |
| | | | Apigenin | |
| | | | Chrysin | |
| | | | Luteolin | |
| | | | Tangeritin | |
| | | Flavonols | | Onions, Green |
| | | | | Onions, Broccoli, |
| | | | | Apples, Hibiscus |
| | | | Isorhamnetin | |
| | | | Kaempferol | |
| | | | Myricetin | |
| | | | Proanthocyanidins | |
| | | | (condensed | |
| | | | tannins) | |
| | | | Quercetin and | |
| | | | related | |
| | | Flavanones | | Citrus Fruits, |
| | | | | Tomatoes |
| | | | Eriodictyol | |
| | | | Hesperetin | |
| | | | Naringenin | |
| | | Flavanols | | Tea, Chocolate, |
| | | | | Grapes, Berries, |
| | | | | Apples |
| | | | Catechin and | |
| | | | related | |
| | | | Epicatechin and | |
| | | | related | |
| | | | Theaflavin | |
| | | | Thearubigins | |
| | | Isoflavone | | Soy beans, Legumes |
| | | Phytoestrogens | | |
| | | | Daidzein | |
| | | | Genistein | |
| | | | Glycitein | |
| | | Stilenoids | | Red Grapes, Berries, |
| | | | | Pomegranate |
| | | | Resveratrol | |
| | | | Pterostilbene | |
| | | Anthocyanins | | Red, Blue & Purple |
| | | | | Berries, Red Wine, |
| | | | | Red Cabbage, Red |
| | | | | Radish, Hibiscus |
| | | | Cyanidin | |
| | | | Delphinidin | |
| | | | Malvidin | |
| | | | Pelargonidin | |
| | | | Peonidin | |
| | | | Petunidin | |
| | | | Beta-cyanin | Beets |
| | Phenolic | | | Berries, Tea, |
| | Acids and | | | Pomegranate, |
| | Esters | | | Mangoes, Spices, |
| | | | | Onions, Corn, Rice, |
| | | | | Oat, Coffee, |
| | | | | Cinnamon |
| | | | Chicoric Acid | |
| | | | Chlorogenic Acid | |
| | | | Cinnamic Acid | |
| | | | and related | |
| | | | Ellagic Acid | |
| | | | Ellagitannins | |
| | | | Gallic Acid | |
| | | | Gallotannins | |
| | | | Rosmarinic Acid | |
| | | | Salicylic Acid | |
| | Other | | | Spices (Turmeric), |
| | Nonflavanoid | | | Grape seed, Cocoa, |
| | Phenolics | | | Mangosteen, Berries |
| | | | Coumarins | |
| | | | Tannins | |
| | | | Curcumin | |
| | | | Flavonolignans | |
| | | | Xanthones | |
| | | | Santalin (Santalic | Red Sandalwood |
| | | | Acid) | |

| Class of | | | | Food/Supplement |
|---|---|---|---|---|
| Compounds | Sub-Class A | Sub-Class B | Compound | Source |
| Vitamins | | | | |
| | Vitamin A | | | Carrots, Dark |
| | (retinol) | | | Green, Yellow and |
| | | | | Orange Vegetables |
| | | | | and Fruit |
| | | Beta-Carotene | | |
| | | Alpha- | | |
| | | Carotene | | |
| | | Gamma- | | |
| | | Carotene | | |
| | Vitamin C | | | Citrus Fruits, |
| | (ascorbic acid) | | | Acerola, Camu- |
| | | | | Camu, Amla, Green |
| | | | | Leafy Vegetables, |
| | | | | Black currants |
| | Vitamin D | | | Fish (especially oil), |
| | (D2 & D3) | | | Mushrooms, |
| | | | | Cheese, Eggs |
| | Vitamin E | | | Nut, Seed & |
| | | | | Vegetable Oils, |
| | | | | Green Leafy |
| | | | | Vegetables |
| | | Tocotrienol | | |
| | | Tocopherol | | |
| | Vitamin K | | | Natto (Fermented |
| | (K1 & K2) | | | Soy) Extract, |
| | | | | Brassicacea |
| | | | | Vegetables, Green |
| | | | | Leafy Vegetables |
| Vitamin Co- | | | | |
| Factors and | | | | |
| Minerals | | | | |
| | | Coenzyme | | |
| | | Q10 | | |
| | | Manganese | | |
| | | Iodide | | |
| | | Ergothionine | | |
| Hormones | | | | |
| | | Melatonin | | |
| Carotenoid | | | | |
| Terpenoids | | | | |
| | | Alpha- | | See Vitamin A |
| | | Carotene | | |
| | | Astaxanthin | | Red Algae |
| | | Beta-Carotene | | See Vitamin A |
| | | Beta- | | Citrus Fruit, Bell |
| | | Cryptoxanthin | | Peppers, |
| | | | | Avocadoes, Paprika |
| | | Canthaxanthin | | Fish, Algae, |
| | | | | Crustacea |
| | | Lutein | | Spinach, Kiwi Fruit, |
| | | | | Red Peppers |
| | | Lycopene | | Tomatoes, |
| | | | | Watermelon |
| | | Zeaxanthin | | Corn, Kiwi Fruit |
| | | Bixin | | Annatto |
| | | Capsanthin, | | Paprika |
| | | Vyolaxanthin | | |
| | | & Capsorbin | | |
| Organosulfur | | | | Garlic, Brassicacea |
| Compounds | | | | Vegetables |
| | | | | (Broccoli), Mustard |
| | | | | Seed, Onion |
| | | Allylic Sulfur | | |
| | | Compounds | | |
| | | Glucosinolates | | |
| | | Indoles | | |
| | | Isothiocyanates | | |
| Other Organic | | | | |
| Antioxidants | | | | |
| | | Chlorophyll | | Green Leafy |
| | | | | Vegetables, |
| | | | | Spirulina, Chlorella |
| | | Bilirubin | | |
| | | | Citric Acid | |
| | | | N- | |
| | | | Acetylcysteine | |
| | | | R-Alpha- | |
| | | | Lipoic Acid | |
| | | | Uric Acid | |

Please see the Antioxidant Family Charts in Appendix A for additional characterization of antioxidant compounds into families. Appendix A is incorporated entirely in this specification by reference.

### Currently Available Natural Colors

### Single Ingredients:

### Grape/Grape Concentrate/Grape Skin

### Beet

### Red Cabbage

### Red Radish

### Hibiscus

### Carmine

### Red Sandalwood

### Purple/Black Carrot

### Purple Sweet Potato

### Purple Corn

### Black Currant

### Bilberry

### Elderberry

### Maqui Berry

### Natural Caretonoids

### Carrot

### Turmeric/Curcumin

### Paprika

### Annatto

### Lutein

### Marigold

### Spinach

### Chlorophyll

### Spirulina

Combination of Ingredients—Any combination of these ingredients is also acceptable.

### Enhanced Natural Colors

### Single Ingredients

### Grape/Grape Concentrate/Grape Skin

### Beet

### Red Cabbage

### Red Radish

### Hibiscus

### Carmine

### Red Sandalwood

### Purple/Black Carrot

### Purple Sweet Potato

### Purple Corn

### Black Currant

### Bilberry

### Elderberry

### Maqui Berry

### Natural Carotenoids

### Carrot

### Turmeric/Curcumin

### Annatto

### Lutein

### Marigold

### Spinach

### Chlorophyll

### Spirulina

Combination of Ingredients—Any combination of these ingredients is also acceptable.

### Defining Typical and Enhanced Ranges

In this disclosure there is broad use of two terms: typical range and enhanced range. Below is a definition for both terms:

Typical range(s)—The typical range refers to the measured ranges (like color density, amount of marker or antioxidant compounds, ORAC values, use rates etc.) for natural color ingredients/products that are currently and typically available in the market today. The range reflects the typically available and typically used color ingredients used in the food, dietary supplement and skin-care industries.
Enhanced range(s)—The enhanced range refers to the measured ranges (like color density, amount of marker or antioxidant compounds, ORAC values, use rates etc.) for natural color ingredients/products that have been concentrated or enhanced to increase levels of antioxidant or anti-inflammatory compounds, ORAC values, as well as have enhanced nutritional benefit.

### Color Density Measurements—Typical and Enhanced Ranges

Color density reading is the primary means for evaluating colors. There are a variety of different means of testing color density based on different commercial standards and the characteristics of the product. The most common accepted method, especially for water-soluble compounds, is to measure OD (Optical Density) by diluting the sample and reading it through UV-VIS (ultraviolet to visual spectrum of light) in a spectrophotometer. In Appendix B and Appendix C are color readings and methods for both the typical range of color products in the market and the enhanced ranges. Also in Appendix D are attached written procedures for the analysis of color density.

See Functional Color Analysis Spreadsheet. Also attached, please find written procedures for testing color density (Appendix D). Appendices A, B, C, and D are hereby incorporated into this specification by reference.

### Marker Compounds—Typical and Enhanced Ranges

As stated earlier, there are numerous compounds and families of compounds that have the dual purpose of natural color and antioxidant/anti-inflammatory (nutritional value). There are also a variety of different methods employed to identify and quantify these compounds. Many of these are commonly accepted or published methods. In Appendix B and Appendix C are marker compound values (and ranges) and methods for both the typical range of color products in the market and the enhanced ranges. Also in Appendix D are attached written procedures for the analysis of marker compounds.

See Functional Color Analysis Spreadsheet. Also attached, please find written procedures for testing marker compounds (Appendix D).

### USDA Database for the Flavonoid Content of Selected Foods:

The link below is to the USDA's website disclosing the contend of Flavonoids in a broad spectrum of foods and which serves as a guideline for the importance of concentrating and enhancing composition so as to enhance the antioxidant properties of the products.

http://www.ars.usda.gov/SP2UserFiles/Place/12354500/Data/Flav/Flav02-1.pdf

### ORAC Values—Typical and Enhanced Ranges

There are a few different methods for analyzing ORAC values and a few variations to the most common method (for determination of water soluble antioxidant capacity—hydrophyllic method). In Appendix B and Appendix C are ORAC values (and ranges) and methods for both the typical range of color products in the market and the enhanced ranges. Also in Appendix D are attached written procedures for the analysis of ORAC values.

See Functional Color Analysis Spreadsheet. Also attached, please find written procedures for testing ORAC value (Appendix D).

### Antioxidants and the Importance of Bio-Assays Like ORAC

Free radicals are highly reactive compounds which cause damage to cellular components such as DNA and cell membranes. Such damage is called “oxidative damage” and is the common pathway in the aging process, inflammation and such diseases as:

Cancer

Diabetes

Arthritis

Cardiovascular Disease

Antioxidants are incredibly important compounds, which effectively “mop up” free radicals produced through metabolism and environmental stresses. ORAC (Oxygen Radical Absorbance Capacity) assay measures the ability of a substance to disarm oxygen free radicals and thereby inhibiting their ability to cause oxidative damage. The ORAC assay compares a sample to Trolox (a non-commercial water-soluble derivative of tocopherol). The results are then reported as μmoles Trolox Equivalents (TE)/g.

This method has become synonymous with antioxidant potency in the dietary supplement industry and in food industry. The ORAC assay can provide a much-needed system to compare the antioxidant capacity of various products to the ORAC intake of healthy diet.

### Oxygen Radical Absorbance Capacity (ORAC) of Selected Foods—2007

The link below is to the USDA's website disclosing the most recent ORAC values for a broad spectrum of foods and which serves as a guideline for the importance of concentrating and enhancing composition so as to enhance the ORAC values of the compounds.
http://www.ars.usda.gov/SP2UserFiles/Place/12354500/Data/ORAC/ORAC07.pdf
Claimed Health Benefits from Enhanced Composition

The enhanced composition of the single and combination products has a variety of health related functions and benefits. The benefits and functions are associated to the antioxidant and anti-inflammatory properties of the enhanced color products. These benefits and functions may include, but are not limited to:

### Cardiovascular Benefits

### Cholesterol and Plaque Reducing Properties

### Immune Enhancing Properties

### Micro-circulatory Benefits

### Cognitive Health Benefits

### Mood Enhancing Properties

### Eye Health

### Anti-Inflammatory Function

### Energy and Endurance Functions

### Hormonal Balancing

### Sexual Function

### Combining Pigments to Enhance Color as Well as Nutritional Properties

It is common practice in the natural colors industry to combine different ingredients to produce different shades and hues with specific applications. This same concept is applied to the discovery with added benefits in addition to the hues. The combination of different nutritional pigments may promote synergistic effects. Anthocyanins, for example, are a fairly large family of compounds, many of which have specific nutritional function, including antioxidant and anti-inflammatory effects. The combination therefore of different nutritional pigments may have health promoting properties. Some examples of these may include, but are not limited to:

### Cardiovascular Benefits

### Cholesterol and Plaque Reducing Properties

### Immune Enhancing Properties

### Micro-circulatory Benefits

### Cognitive Health Benefits

### Mood Enhancing Properties

### Eye Health

### Anti-Inflammatory Function

### Energy and Endurance Functions

### Hormonal Balancing

### Sexual Function

The combination of these pigments can take place in a variety of different ways, but ideally constitutes blending the different sources of the pigments to produce color and health enhancing properties. All of the color sources listed herein can be mixed and blended to further enhance the nutritional benefits of the blend.

The Relationship of Color Pigments with other Endogenous Compounds in the Matrix

Most of the color ingredients contain antioxidant compounds and these compounds have been concentrated or enhanced to produce the nutritional color products. In many of these cases, the antioxidant compounds are part of a matrix of other compounds that comprise the product. The matrix comprises other endogenous compounds like water, carbohydrates (a variety of different sugars), fats, protein, vitamins and minerals. These other endogenous compounds also perform an important role in the performance of the products, either as stabilizing agents (compounds that help protect the other active ingredients in the matrix) or as supporting nutritional compounds. This is certainly the case of vitamins, minerals, some fats and proteins. It is also true that many of the sugars, especially the complex sugars in some of these products can act as transport compounds. This means these compounds can enhance the delivery of the antioxidant compounds into the blood stream and as a result improve bioavailability as well as enhance the properties of these nutritional colors.

The Relationship of Color Pigments with Other Exogenous Compounds in the Matrix

The discovery also recognizes the importance of exogenous, or added, ingredients to the product matrix. There are primarily two types of Exogenous Compounds that go into the matrix: those added to the product to aid in stability or other color function, and those added to the matrix to enhance nutritional properties. This discovery claims that there are many exogenous compounds that fit these purposes and that these compounds may also enhance, preserve, or otherwise support the nutritional value of the pigments.

Exogenous Compounds that aid Stability or other Color Function, including but not limited to:

### Rosemary

### BHT

### Citrus Oils

### Citric Acid

### Potassium Sorbate

The discovery also claims that exogenous compounds can be added to the matrix that enhance antioxidant, anti-inflammatory or other nutritional qualities to the matrix. Exogenous Compounds that Enhance Nutritional Properties include, but are not limited to:

All the antioxidants listed in this disclosure, especially under the section “Family of Compounds”

Heart and Cardiovascular health promoting compounds including, but not limited to: Co-enzyme Q10 (CoQ10), Resveratrol, statins, phytosterols, dietary fiber, polysaccharides

Energy compounds including, but not limited to: methylxanthine alkaloids (caffeine, theobromine and theophylline) along with their natural sources (coffee, tea, green tea, white tea, mate, cocoa, kola nut)

Mood enhancing compounds including, but not limited to: L-theanine, amino acids (especially L-arginine, L-Phenylalanine, L-Tryptophan), Rhodiola rosea, Cognitive enhancing compounds including, but not limited to: Omega 3 Fatty Acids (from Fish and Vegetable oils), DHA & EPA, Phosphatidyl Choline, Phosphatidyl Serine, Gingko Biloba

It will be apparent to one with skill in the art that the natural color of the invention may be provided using some or all of the mentioned features and components without departing from the spirit and scope of the present invention. It will also be apparent to the skilled artisan that the embodiments described above are specific examples of a single broader invention that may have greater scope than any of the singular descriptions taught. There may be many alterations made in the descriptions without departing from the spirit and scope of the present invention.

What is claimed is:

1. A natural color concentrated, intensifying color range and providing enhanced levels of one or more of anti-oxidant, nutritional, and anti-inflammatory compounds, the natural color derived from one or more pigment sources.

2. The natural color of claim 1, wherein the pigment source is a fruit, a vegetable, a legume, a spice, an algae, or a combination thereof.

3. The natural color of claim 1, wherein the pigment or pigments in the color are extracted from or concentrated from one or a combination of grape, beet, red cabbage, red radish, hibiscus, carmine, red sandalwood, purple carrot, black carrot, purple sweet potato, purple corn, black currant, bilberry, elderberry, maqui berry, natural carotenoids, carrot, turmeric, curcumin, paprika, annatto, lutein, marigold, spinach, chlorophyll, and spirulina.

4. The natural color of claim 1, wherein the pigment or pigments in the color are extracted by one of or a combination of screw press, hydraulic press, juicing, natural solvent extraction, synthetic solvent extraction, and distillation.

5. The natural color of claim 4, further processed by one or a combination of vacuum concentration, steam concentration, supercritical carbon dioxide extraction, distillation, ultra-filtration, membrane filtration, column purification, and ion exchange.

6. The natural color of claim 1, wherein the color compound is dried using one or a combination of spray drying, vacuum drying, drum drying, refractance window drying, radiant zone drying and freeze drying.

7. The natural color of claim 1, wherein the color compound is endogenous.

8. The natural color of claim 1, wherein the color compound is exogenous.

9. The natural color of claim 7, wherein the color compound includes one or more of vitamins, minerals, fats, proteins, and sugars.

10. The natural color of claim 8, wherein the color compound includes one or a combination of rosemary, butylated hydroxytoulene (BHT), citrus oils, citric acid, and potassium sorbate.

11. The natural color of claim 8, wherein the color compound includes one or more of the compounds co-enzyme Q10 (CoQ10), resveratrol, statins, phytosterols, and dietary fiber.

12. The natural color of claim 8, wherein the color compound includes one or more of polysaccharides, methylxanthine, caffeine, theobromine and theophylline.

13. The natural color of claim 8, wherein the color compound includes one or more of 1-thiamine, 1-arginine, 1-phenylalanine, 1-tryptophan, rhodiola, and rosea.

14. The natural color of claim 8, wherein the natural color includes one or more of omega 3 fatty acids, docosahexanoic acid (DHA), eicosapentaenoic acid (EPA), phosphatidyl choline, phosphatidyl serine, and gingko biloba.
